(19) United States
(12) Patent Application Publication
(10) Pub. No.: US 2026/0294267 A1
(43) Pub. Date: Oct. 1, 2026

(54) MEDICAL DEVICE TRACKING WITH SHORTEST DISTANCE DETECTION

(71) Applicant: DEEPSIGHT TECHNOLOGY, INC., Santa Clara, CA (US)

(72) Inventors: Jiangang Zhu, St. Louis, MO (US); Sitai Kou, Brentwood, MO (US); Thomas Mark Horning, St. Louis, MO (US); Robert Steins, Sausilito, CA (US); Timothy Hudson Mason, Brentwood, MO (US); Basile Hurat, Mountain View, CA (US); John Matthew Chong, Ithaca, NY (US); Lan Yang, Clayton, MO (US)

(73) Assignee: DEEPSIGHT TECHNOLOGY, INC., Santa Clara, CA (US)

(21) Appl. No.: 19/631,141

(22) Filed: Mar. 27, 2026

Related U.S. Application Data
(60) Provisional application No. 63/779,612, filed on Mar. 28, 2025.

(51) Int. Cl. A61B 5/06

(52) U.S. Cl. CPC A61B 5/061

(57) ABSTRACT

Systems and methods for tracking a medical device and ultrasound imaging with an optical sensor in a medical procedure are provided as described. A processor receives sensor data from a sensor coupled to the medical device. The sensor receives one or more acoustic signals in the body region and converts the acoustic signals to the sensor data. The processor determines a set of time-of-flight (ToF) data associated with the sensor based on the sensor data. The processor determines a location of the sensor based on the ToF data set and multiple sets of pre-determined ToF data associated with multiple locations in the body region.

### CROSS-REFERENCE TO RELATED APPLICATIONS

This application claims priority from U.S. Provisional Patent Application No. 63/779,612, filed Mar. 28, 2025, titled “MEDICAL DEVICE TRACKING WITH SHORTEST DISTANCE DETECTION,” the entirety of which is hereby incorporated by reference.

### FIELD OF THE INVENTION

The present disclosure relates to optical sensing, and without limitation to medical device tracking with shortest distance detection and ultrasound imaging with an optical sensor.

### DESCRIPTION OF RELATED ART

Acoustic or ultrasound imaging technology is used in various industries, particularly in non-invasive measurements, remote sensing, and medical imaging. Acoustic imaging technology operates by transmitting acoustic signals toward an object and detecting resulting echo signals that reflect or generate from the object in response to the transmitted acoustic signals. Ultrasound is an advantageously non-invasive form of imaging. Ultrasound imaging allows viewing of soft tissues and surrounding anatomy. Ultrasound imaging may also be used to view the location of various medical devices in situ, such as needles, scopes, catheters, interventional tools and temporary or permanent indwelling devices.

### SUMMARY

Various examples and embodiments are described in relation to medical ultrasound device tracking with shortest distance detection and ultrasound imaging with an optical sensor. For example, a processor obtains sensor data from a sensor coupled to the medical device in a body region. The sensor receives one or more acoustic signals and converts the acoustic signals to the sensor data. The one or more acoustic signals are emitted by a transducer. The processor determines a set of time-of-flight (ToF) data associated with the sensor based on the sensor data. The processor determines a location of the sensor based on the ToF data set and multiple sets of pre-determined ToF data associated with multiple locations in the body region.

These illustrative examples are mentioned not to limit or define the scope of this disclosure, but rather to provide examples to aid understanding thereof. Illustrative examples are discussed in the Detailed Description. Advantages offered by various examples may be further understood by examining this specification.

### BRIEF DESCRIPTION OF DRAWINGS

The accompanying drawings, which are incorporated into and constitute a part of this specification, illustrate one or more certain examples and, together with the description of the examples, serve to explain the principles and implementations of the certain examples.

FIG. 1 is an example of a system for ultrasound visualization and tracking of a medical device, according to this disclosure.

FIG. 2 is an elevation view of an ultrasound probe and a sensor coupled to a medical device in a biopsy procedure, according to this disclosure.

FIG. 3 shows an example method of tracking the location of a medical instrument during a medical procedure, according to this disclosure.

FIG. 4 shows an optical sensor coupled to a needle receiving a reflected ultrasound signal from a tissue spot, according to this disclosure.

FIG. 5 shows an ultrasound image depicting the view of a body region from the perspective of a probe and an OnPoint image depicting the view of the body region from the perspective of a sensor in the body region, according to this disclosure.

FIG. 6 shows an example method of reconstructing an ultrasound image to generate an OnPoint image, according to this disclosure.

FIG. 7 shows an example method of time-of-flight calculation for OnPoint image reconstruction.

FIG. 8 shows a B-mode image and a ToF image in a first instance during an example medical procedure, according to this disclosure.

FIG. 9 shows a B-mode image and a ToF image in a second instance during an example medical procedure, according to this disclosure.

FIG. 10 shows a B-mode image and a ToF in a third instance during an example medical procedure, according to this disclosure.

Similar reference characters denote corresponding features consistently throughout the attached drawings.

### DETAILED DESCRIPTION

Examples are described herein in the context of medical device tracking and position determination with shortest distance detection, and imaging from the perspective of the medical device while in situ. Those of ordinary skill in the art will realize that the following description is illustrative only and is not intended to be in any way limiting. Reference will now be made in detail to implementations of examples as illustrated in the accompanying drawings. The same reference indicators will be used throughout the drawings and the following description to refer to the same or like items.

In the interest of clarity, not all of the routine features of the examples described herein are shown and described. It will, of course, be appreciated that in the development of any such actual implementation, numerous implementation-specific decisions can be made in order to achieve the developer's specific goals, such as compliance with application-and business-related constraints, and that these specific goals will vary from one implementation to another and from one developer to another.

The following U.S. Patent Applications are related to the present disclosure: U.S. application Ser. No. 19/403,738, filed Nov. 28, 2025, titled “Fiber Sensors for Interventional Tools;” U.S. application Ser. No. 18/597,493, filed on Mar. 6, 2024, titled Mixed Array Imaging Probe;” U.S. application Ser. No. 17/990,596, filed on Nov. 18, 2022, titled “Mixed Ultrasound Transducer Arrays;” U.S. application Ser. No. 17/244,605 filed on Apr. 29, 2021, titled “Modularized Acoustic Probe;” U.S. application Ser. No. 18/492,593, filed on Oct. 23, 2023, entitled “FIBER OPTICAL SENSOR SYSTEM FOR ULTRASOUND SENSING AND IMAGING;” U.S. application Ser. No. 18/382,984, filed Oct. 23, 2023, entitled “TRANSPONDER TRACKING AND ULTRASOUND IMAGE ENHANCEMENT;” U.S. Provisional Application No. 63/779,440, filed on Mar. 28, 2025, titled “Medical Devices with Integrated Ultrasound Sensors;” and U.S. Provisional Application No. 63/779,812 filed on Mar. 28, 2025, titled “Fiber-Optical Sensor System for Facilitating Ablation Procedures.”

Object visualization, tracking, and location in medical applications are important aspects for performing medical procedures in a safe and reliable manner. Therapeutic and diagnostic medical applications include ultrasound imaging as well as sensing (e.g., tracking, visualizing, and monitoring) of objects (e.g., needles, introducers, catheters, guidewires, etc.) during guided needle access, biopsy, aspiration, delivery of drugs, biologics, anesthesia or other therapeutics, catheterization, minimally invasive procedures, ablation, cauterization, placement or moving of objects, tissue, cutting, sectioning, and other medical procedures. Procedures and applications in the following disciplines are examples of the wide usage and need for accurate guidance and imaging during diagnostic, monitoring and therapeutic procedures: anesthesia, cardiology, critical care, dermatology, emergency medicine, endocrinology, gastroenterology, gynecology and obstetrics, hepatology, infectious diseases, interventional radiology, musculoskeletal medicine, nephrology, neurology, oncology, orthopedics, pain management, pediatrics, plastic and reconstructive surgery, urology, vascular access, and other disciplines. In non-medical applications, ultrasound is used in industrial applications for defect detection and microparticle particle sorting among other applications, non-destructive testing, structural testing, geological applications including mining and drilling operations, and underwater marine applications. Such applications are consistent with the embodiments described herein.

Objects for tracking, visualization, and determining location may include any type of medical device that travels or is located within or close to the body of a subject. For instance, medical practitioners visualize and track a needle tip while conducting a biopsy to ensure safety and ensure the intended tissue is biopsied. In such instances, accurate needle tip visualization or tracking may help to prevent or reduce unintentional vascular, neural, tissue or visceral injury. This technique can help predict the needle's trajectory, ensuring it reaches the targeted area as planned and repeat procedures may be prevented because sufficient tissue of the biopsy target was obtained on the initial biopsy due to accurate tracking of the needle. Similarly, it may be helpful to visualize, track, or locate needles, introducers, endoscopes, cannulas, laparoscopic tools or other medical device tools when performing medical procedures such as, but not limited to, aspiration of fluid; injections of joints, tendons, and nerves with drugs or biologics; biopsy of fluids or soft tissue masses; aspiration and lavage of calcifications; removal of tissue, organs or foreign bodies, placement of a stent, filter, valve, permanent, temporary or biodegradable implant, shunt or drain, transcatheter procedures including but not limited to Transcatheter Aortic Valve Replacement (TAVR), Transcatheter Mitral Valve Replacement (TMVR), and Transcatheter Edge-to-Edge Repair (TEER), injections for anesthesia, inserting vascular access devices used for infusion therapies, ablation procedures, performing the Seldinger technique or catheterization to gain access to blood vessels and/or other organs in a safe manner. Visualization and tracking may be advantageous in minimally invasive surgical, robotic surgical, interventional, robotic interventional and open surgical procedures, especially when the area of interest is hidden or obstructed by tissue, blood or fluids.

In an additional embodiment, the ultrasound guided sensor tracking of the present invention may supplement and/or compliment other positional tracking and imaging technologies (inertia measurement unit (IMU), robotic, electromagnetic (EM), optical, Computed Tomography (CT), Magnetic Resonance Imaging (MRI), x-ray, fluoroscopy etc.) to provide real-time tissue positional reference which may not be available or optimized in such devices. Also, being able to generate an ultrasound image from a passive optical sensor in situ may eliminate or reduce the need for using other imaging technologies during the procedure.

Example systems and methods according to this disclosure address a critical gap in many minimally invasive procedures where tool motion becomes unpredictable after deployment (like a semi-automatic or automatic biopsy needle firing into the prostate to obtain tissue for a cancer diagnosis or device insertion into soft tissue). Furthermore, certain anatomies can be very challenging due to the close proximity of critical anatomy such as the spinal cord, thyroid, and others without limitation, or the presence of strong ultrasound reflectors near the target (such as the diaphragm). Lastly, during procedures such as ablation, a thermal cloud and/or tissue changes created during the procedure can be problematic to imaging with conventional technologies. This system can help with pre-firing planning, trajectory prediction, and real-time guidance, especially in procedures with limited visibility, thermal clouds, tissue changes during the procedure or tissue deformation during the procedure. Example systems and methods according to this disclosure provide real-time guidance and predictive modeling for minimally invasive tools, such as biopsy needles, by integrating intra-procedural imaging with trajectory prediction algorithms that account for tissue resistance and anatomical variability. The system enables physicians to visualize and track the position and projected path of tools within soft tissues, even after active deployment (e.g., firing of a spring-loaded biopsy needle), where manual control is no longer feasible. By incorporating prior knowledge of tool-tissue interaction—including empirical data or models of how tools decelerate or deviate upon entering biological tissue—example systems and methods can optimize the initial position, orientation, deployment and other procedural parameters to ensure accurate targeting and in situ imaging of the anatomical regions of interest. This technology applies broadly to interventional procedures including, but not limited to, prostate biopsy, liver or kidney interventions, spinal injections, nephrostomy procedures, ablation procedures and tumor sampling, and may be adapted for use with CT, MRI, or other imaging modalities. This technology offers capabilities to enhance precision, reduce procedural risk, and improve diagnostic yield by combining pre-procedural planning with intraoperative decision support, forming a closed-loop system for image-guided intervention.

In an example ultrasound guided intervention system, an ultrasound transponder is coupled to a medical device (e.g., a needle) that is to be inserted into the tissue or body lumen of a patient (e.g., a human or animal). The transponder may be an ultrasound receiver or transmitter or a combination of both. An example transponder includes a sensor. In some examples, the sensor is a fiber optical sensor. The sensor can be a point sensor, a line sensor, or a sensor formed in some other known shape. The sensor may be coupled to one end of the needle, such as the distal end of a needle, which is the end that first penetrates the tissue or enters a body cavity or lumen.

An example system may also include an inertia measurement unit (IMU) coupled to the medical device. The IMU includes one or more accelerometers configured to measure linear acceleration along each axis and one or more gyroscopes configured to measure rotational position or angular velocity around each axis. Thus, IMU sensor data can be used to track the position and motion of the medical device coupled with the optical sensor.

An example system also includes an ultrasound probe. The ultrasound probe includes an array of transducers that output and receive a plurality of acoustic beamforming pulses or signals. The probe may be any one of the known probes such as a hand-held probe, EBUS (endobronchial ultrasound), EUS (endoscopic ultrasound), IVUS (intravascular ultrasound), ICE (intracardiac echocardiogram), TEE (transesophageal echocardiogram), an ultrasound patch or any acceptable ultrasound emitter. An example system also includes associated electronics for receiving data from the ultrasound probe, the transponder, and the IMU. An example system also includes a computer processor for utilizing and processing the data for generating an ultrasound image and tracking the medical device in the patient. An example system also includes a display for visualizing the body region for the medical procedure and the location of the medical device.

While ultrasound-guided tracking is easy to set up and has a high accuracy level, it faces certain challenges. For example, there is a lack of clear guidance for physicians on where and at what angle to insert a medical device or instrument (e.g., a needle or introducer for receiving other procedural instruments) for an ultrasound-guided medical procedure. Also, once the needle introducer, or other instrument is inserted, only small positional or angular adjustments are possible, otherwise reinsertions may be needed for larger adjustments. However, repeated insertions increase tissue damage and may cause patient discomfort after the procedure. In addition, the current tracking techniques are most effective or accurate when the instrument is in the imaging plane, which is referred to as being “in-plane.” When the instrument is not in-plane (which is can be referred to as “out of plane”), or has not been inserted into the patient, the current tracking techniques may not be able to accurately track the needle or otherwise provide accurate trajectory information. This may result in more instrument maneuvers over a longer period of time, which increases risks of damaging tissue and medical procedure failure. Thus, there is a need for accurate location detection to provide accurate trajectory planning before the instrument is inserted into the skin and for accurate tracking during the medical procedure.

Instrument tracking methods include time-of-flight (ToF) methods, triangulation methods or coherent imaging (including coherent beamforming) which involves detecting bright spots in an ultrasound image produced using signals generated by the optical sensor. The coherent image method may be prone to reflection and scattering, which is common in real tissues. These methods work well when the optical sensor received signal has a strong signal to noise (SNR) and/or signal strength (SS). However, the SNR/SS will vary as the sensor moves across the skin or within the tissue during the procedure. The SNR/SS is strongest when the sensor is in the ultrasound imaging plane (in-plane) and is not surrounded by strong reflections or scattering from anatomical (and/or device) features in the area of needle insertion.

To overcome these shortcomings, the present tracking technique detects the shortest distance from the transducer to the optical sensor to determine the location of the optical sensor coupled to the needle. The shortest distance method is applicable even when the optical sensor is far outside of the imaging plane, for example when the needle contacts the surface of the skin. In the present disclosure, the computer processor uses or implements a shortest-distance algorithm to accurately determine the location of the sensor coupled to the medical device for trajectory planning and tracking.

Prior to a medical procedure, the computer processor may develop a three-dimensional (3D) anatomical model of a region where the medical procedure is needed and identify the location of a target for the medical procedure, using ultrasound sensing or other technologies. In some examples, the 3D model of the region and the target location are pre-determined by a third-party system. The 3D model and the target location-related data are provided to the computer processor. The target location can be visualized in the 3D anatomical model of the region.

During the medical procedure, the probe is in contact with the skin surface in the region where the medical procedure is needed and transmits acoustic beamforming signals into the region. As soon as the needle touches the skin surface, the optical sensor coupled to the needle receives acoustic signals. The optical sensor then converts the received acoustic signals to optical signals, which may be converted to electrical signals before being transmitted to the computer processor. The IMU coupled to the needle collects sensor data, such as orientation, to transmit to the computer processor, such as via wired communications, such as USB, or via certain wireless communication protocol, for example Bluetooth, Near Field Communication (NFC), or Radio Frequency Identification technology (RFID). The computer processor determines the location based on the data from the optical sensor using the shortest distance method and determines the orientation and projected trajectory of the medical instrument. The projected path trajectory can be determined based on a path history, which is a feature history representing the previous locations of the sensor. The projected path trajectory can be depicted on the screen as a line representing a “best fit” obtained by curve analysis techniques based on recent and current locations. The location and orientation of the medical device on the skin surface may be adjusted with respect to the target so that an optimized trajectory can be determined. The computer processor then determines the optimized trajectory from the medical device to a target location within the body. The target location can be the location of a tumor that needs biopsy. When the medical device is inserted through the skin surface, the probe continues to transmit ultrasound signals, and the optical sensor coupled to the medical device receives acoustic signals once it enters the tracking region. The computer processor continues to determine the location of the medical device by detecting the shortest distance between the probe transducer and the optical sensor coupled to the medical device (e.g., the tip of a needle). The shortest distance between the probe transducer and the optical sensor may be determined by ToF, triangulation, or coherent image formation. The location of the optical sensor can then be used in conjunction with the ultrasound image, e.g., the sensor location overlayed on the ultrasound image, to track and visualize the location of the medical device with respect to the target location. For example, as a medical practitioner moves or adjusts the needle, a display shows the location of the needle and its projected trajectory toward the target.

Example systems and methods according to this disclosure allow for an optimized insertion location and angle by trajectory planning before a medical device is inserted into the body. Once the medical device is inserted, only small positional adjustments may be needed. Thus, it reduces tissue damage and related complications related to mispositioning. In addition, by detecting the shortest distance between the optical sensor and the probe transducer, example systems and methods according to this disclosure accurately determines the location of the medical device for tracking. Thus, the medical device can be navigated to the target accurately.

This illustrative example is given to introduce the reader to the general subject matter discussed herein and the disclosure is not limited to this example. The following sections describe various additional non-limiting examples of medical device tracking with shortest distance detection.

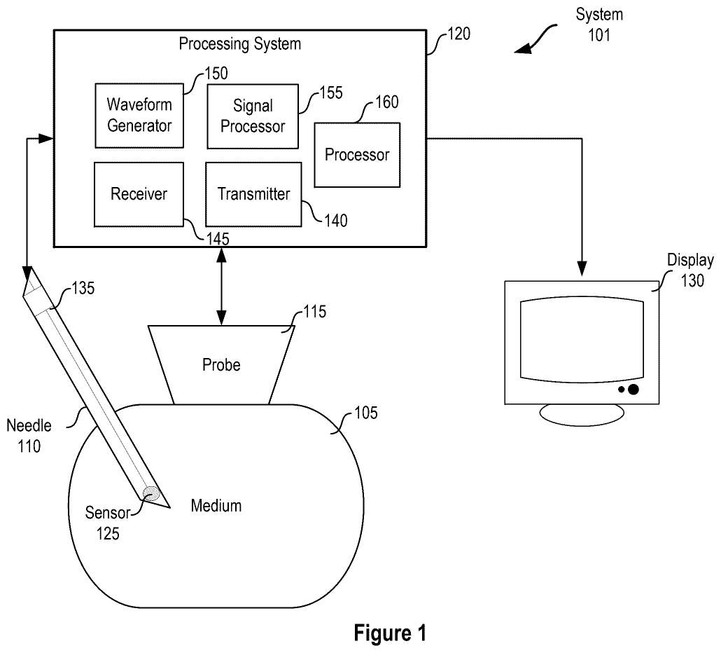

Turning to FIG. 1, FIG. 1 is an example of a system 101 for ultrasound visualization and tracking of a medical device, along with providing an OnPoint ultrasound images from the perspective of the sensor disposed on the medical device. In FIG. 1, the medical device is a needle 110 present in a medium 105 (e.g., body tissue, body cavity, body lumen). However, it should be understood that in other instruments may be used in some examples of the system 101, which may be used for ultrasound visualization and tracking of other medical devices such as a catheter, an ablation tool, nephrostomy tube, a guidewire, an intravenous (IV) line, an endoscope, a trocar, an implant, combinations thereof. System 101 may also be used to enhance visualization of aspects present in the medium 105, such as, for example, organs, vessels, tissue, tumors, other anatomical structures, other medical devices, or implants by providing ultrasound images from the perspective of the sensor within the medium. Further, while the examples that follow describe imaging and determining the location of a medical device, examples of this disclosure may be utilized to locate non-medical devices as well, such as applications in non-medical industries that use ultrasound-based imaging and/or tracking and/or spectroscopy.

In some examples, the system 101 may comprise a processing system 120 in communication with an ultrasound probe 115, a transponder in the form of an optical sensor 125 coupled to a needle 110, and a display 130. In some examples, the needle 110 may comprise more than one sensor 125 or combinations of sensors 125 on a distal end that is inserted into the medium 105. While the sensor 125 is shown in FIG. 1 as a single element, there may be separate multiple elements arranged adjacent or spaced apart from each other or in an array form factor. In some examples, the needle 110 also includes an IMU 135, for example on the other (proximal) end of the needle 110. However, the IMU 135 can be coupled or fused to any part along the needle 110. The IMU 135 includes one or more accelerometers and one or more gyroscopes. During a procedure, the needle 110 may be inserted into the medium 105 and moved independently from motion of the probe 115.

In use, the probe 115 may be placed adjacent to the medium 105 (e.g., placed externally over body tissue) to emit (transmit) and receive ultrasound pulses, which may also be referred to as ultrasound beamforming signals. The area of the medium receiving the ultrasound beamforming signals may be referred to as a directly insonified region. In some examples, the probe 115 is in vivo, such as intravascular ultrasound (IVUS), endobronchial ultrasound (EBUS), endoscopic ultrasound (EUS) (e.g., tracking a needle or other device that extends out of the distal end of a catheter or endoscope such as for biopsy), intracardiac echocardiogram (ICE) or transesophageal echocardiogram (TEE). In some examples, the probe 115 includes an ultrasound array with one or more elements (e.g., transducers) to output (e.g., generate) acoustic pulses and/or receive acoustic signals (e.g., echo signals) corresponding to the acoustic pulses. For example, the ultrasound array may include one or more elements (e.g., transducers) configured to emit a set of beamformed acoustic pulses (e.g., ultrasound signals) and/or receive a set of beamformed acoustic signals (e.g., ultrasound echoes) corresponding to the set of beamformed acoustic pulses. The set of beamformed signals that correspond to the set of beamformed pulses may be used to generate ultrasound images. In some examples, the medium 105 is a non-linear medium, for example a body tissue. In some examples, the transducer is a linear array on the distal facing end of the catheter that is angled in close proximity to, e.g., abutting, the body tissue. The array may include acoustic energy generating (AEG) elements arranged side-by-side in a linear, annular, or convex configuration to form the array that may be front or side firing, as are arrays for EBUS, IVUS, EUS, ICE and TEE devices.

In some examples, the elements of the probe 115 may be arranged as an array such as an ultrasound array. For example, probe 115 may include one or more acoustic energy (AE) transducers, such as one or more of a piezoelectric transducer, a lead zirconate titanate (PZT) transducer, a polymer thick film (PTF) transducer, a polyvinylidene fluoride (PVDF) transducer, a capacitive micromachined ultrasound transducer (CMUT), a piezoelectric micromachined ultrasound transducer (PMUT), a photoacoustic transducer, a transducer based on single crystal materials (e.g., LiNb03(LN), Pb(Mg113Nb213)—PbTiQ3 (PMN-PT), and Pb(In112Nb112)—Pb(Mg113Nb213)PbTiQ3 (PIN-PMN-PT)), combinations thereof, and the like. It should be understood that the probe 115 may include a plurality of any of the transducer types. In some examples, the ultrasound array includes the same type of elements. Alternatively, the ultrasound array may include different types of elements. The probe 115 can be a traditional ultrasound probe with an acoustic energy generating transmitter and receiver. Alternatively, the probe 115 can be an acoustic-optical probe. In some examples that include the acoustic-optical probe, an ultrasound array includes one or more optical sensors, such as an interference-based optical sensor, which may be one or more of an optical interferometer, optical cavity, optical resonator (e.g., whispering gallery mode (WGM) resonators among others), birefringent sensor, or an optical fiber end facet with an acoustic-responsive structure.

In some examples, the probe 115 is configured to receive beamformed acoustic signals reflected in response to interactions of the beamformed acoustic pulses with the objects present in the medium 105, with the medium 105, and/or with the needle 110. The probe 115 is configured to transmit to the processing system 120 signals corresponding to the received acoustic beamforming signals.

One or more optical sensors 125 are arranged at or near the distal end of the needle 110, as discussed above, and may be configured to receive acoustic signals corresponding to the acoustic pulses emitted by the transducers of the probe 115. The optical sensors 125 convert received acoustic signals into optical signals, which are then transmitted to the processing system 120 via an optical fiber or other suitable waveguide. The fiber optical sensors 125 may be disposed at the end of an optical fiber, adjacent an end of an optical fiber, or at a diagnostic or therapeutic relevant location on the medical device to create a sensor fiber. These fiber optical sensors can be point sensors or line sensors. The fiber optical sensors include resonant structures, including, but not limited to Fabry-Perot (FP) resonators, WGM resonators, optical cavity, and photonic crystal resonators; interferometers, including, but not limited to Mach-Zehnder interferometer (MZI), phase-shift coherent interferometers, and self-mixing interferometers; acoustic induced birefringent polarization sensors; fiber end facets with acoustic responsive structures such as metasurfaces including patterns of small elements arranged to change the wavefront shape of the acoustic signals and maximize the collection of acoustic signals, low-dimensional materials with special optomechanical features that more prone to deformation; and plasmonic structure patterned to amplify light-matter interactions.

In addition to operating as an optical sensor, the fiber end facet structures can also be added to the other fiber optical sensors to further enhance acoustic response. These optical structures are configured to respond to acoustic (such as ultrasound) signals. Reponses to acoustic signals in interference-based fiber optical sensors may be due to the photo-elastic effect and/or physical deformation of the structures. When subject to acoustic signals, the resonant structures, or interferometer structures or fiber end facets with acoustic responsive structures, are subject to mechanical stress and/or strain from the alternating pressures of the acoustic signal sound waves. This mechanical stress and/or strain may change the optical properties of the optical sensor structures due to the photo-elastic effect and may also cause changes or deformations in the physical structure of resonator. With polarization-based sensors, the polarization changes when the light is subjected to acoustic signals. When coupled to a light source (e.g., a laser light source, a broadband light source (e.g., a lamp or LED) or other suitable light source) via an optical waveguide (e.g., an optical fiber), the effect of acoustic signals on the optical sensor structures may be measured due to changes in the light returned from the optical sensor structures via the optical waveguide.

A fiber-based acoustic transducer can be integrated with the optical sensors 125 to further enhance its response to acoustic waves carrying information of the targets of interest. Instead of (solely) relying on external acoustic transducers to emit tracking signals, this design enables in situ acoustic wave generation from a transducer bundled with the tool itself, in close proximity to the tracking sensor. Bundling the acoustic source with the tracking sensor provides several critical advantages. First, SNR/SS is significantly improved due to reduced acoustic attenuation and scattering losses that typically occur when waves must propagate from external sources through heterogeneous tissue layers. Second, geometric alignment and coupling between the acoustic source and sensor is optimized, resulting in higher fidelity and more reliable ToF or Doppler measurements for tracking tool movement. Third, this configuration eliminates spatial uncertainty and angular misalignment common in external setups, allowing for more precise localization of the tool and its tip, especially in deep or complex anatomical regions. Fourth, it supports miniaturization and portability, making the system ideal for compact robotic or handheld platforms used in image-guided procedures. Finally, this co-location of acoustic emitter and sensor facilitates closed-loop feedback, enabling real-time motion compensation or control adjustments during procedures such as needle insertion, lesion targeting, or sample collection. The integration of an acoustic transducer with the minimally invasive tool itself can enhance localization accuracy, improve procedural safety, and enable new applications in precision diagnostics and intervention.

The IMU 135 is coupled or fused to the proximal end of the needle 110. The IMU includes one or more accelerometers configured to measure linear acceleration along each axis and one or more gyroscopes configured to measure rotational rates and angular velocity around each axis. Sensor data from the IMU 135 are transmitted to the processing system to determine the orientation of the needle 110.

In some examples, the processing system 120 may include a transmitter 140, a receiver 145, a waveform generator 150, and one or more processors (e.g., a signal processor 155 and processor 160). The waveform generator 150 may be configured to generate a set of digital waveforms for acoustic beamforming pulses. One or more processors (e.g., processor 160) included in the processing system 120 may be configured to control the waveform generator 150. The waveform generator 150 may be configured to generate and send the digital waveforms to one or more of the transmitters 140 and/or a matched filter/Weiner filter (not shown).

The processor 160 is configured to generate an ultrasound image based on the received acoustic beamforming signals. The received beamforming signals may be those received by the probe 115 and/or sensor 125. The processing system 200 is also configured to analyze the optical signals received from the sensor 125 to determine the location of the sensor, which is also the location of the distal end (e.g., tip) of the needle 110 in the medium 105. Furthermore, the processing system can be configured to analyze the optical signals received from the sensor 125 to create ultrasound images (e.g., OnPoint images) from the perspective of the sensor. The ultrasound images and the location indicator may then be displayed on the display 130.

Although the medical device in FIG. 1 is shown to be a needle 110, it should be understood that another suitable medical device may be visualized and/or tracked using the system 101. For example, system 101 may be used to visualize and/or track various diagnostic, therapeutics and surgical medical devices, such as, but not limited to a catheter, guidewire, needle, endoscope, ablation tool, cauterization tool, vacuum or suction tool, tool for grabbing or moving tissue or other objects, forceps, cutting tool, minimally invasive surgical tool, temporary or permanent implantable devices (ports, stents, valves, valve repair clips) and/or open surgical tools, as the device is advanced into and/or manipulated within the medium, which may include a blood vessel, organ, tissue, cavity, and/or lumen.

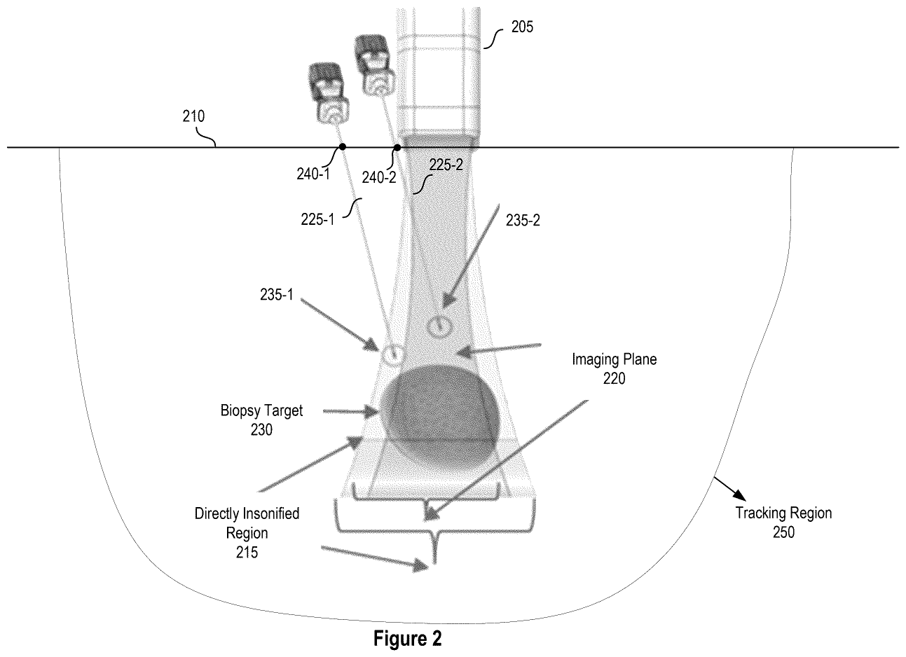

Turning to FIG. 2, FIG. 2 is an elevation view of an ultrasound probe and a sensor coupled to a medical device in a biopsy procedure. The ultrasound probe 205 includes one or more transducers, similar to the probe 115 in FIG. 1. The ultrasound probe 205 contacts a skin surface 210 while the one or more transducers of the ultrasound probe 205 transmit beamformed ultrasound signals to create a directly insonified region 215 under the skin surface 210. The body tissue in the directly insonified region 215 is exposed to beamformed ultrasound signals. The center part of the directly insonified region 215 generally has stronger ultrasound beamformed signals for ultrasound imaging, and the peripheral region of the directly insonified region 215 has weaker beamformed signals for ultrasound imaging. An imaging plane 220 is an imaginary approximately two-dimensional (2D) region through which the beamformed ultrasound signals from the ultrasound probe 205 pass, and is defined by the spatial relationship between the probe 205 and the structure being imaged. The imaging plane has a depth and width, but may also have a small thickness relative to the depth or width dimensions, and thus may be approximately 2D. In FIG. 2, the probe 205 is positioned on the skin surface 210 and transmits beamformed ultrasound signals perpendicularly to the skin surface and down into the body tissue under the skin surface 210. The imaging plane 220 in FIG. 2 is a vertical slice or plane in the body tissue under the probe 205 where the beamformed signals pass through. It is to be understood that the imaging plane may be 3D, 4D or even 5D depending upon the type of ultrasound probe configuration. The directly insonified region 215 and the imaging plane 220 are defined usually for purposes of ultrasound imaging. A tracking region 250 is a region under the skin surface 210 or on the skin surface 210 itself where acoustic signals can be detected and used for tracking the sensor 235 coupled to the needle 225. The acoustic signals not only include the beamformed acoustic signals received directly from the transducer of the probe 205, but also include acoustic signals reflected or scattered by body tissue at or under the skin surface 210. The tracking region 250 is a much larger region than the directly insonified region 215. If the beamformed ultrasound signals are strong enough and a portion of the beamformed signals from the probe 205 can be scattered to different directions, the entire body of a patient can be the tracking region.

In some examples, it may be desirable to intentionally direct or scatter beamformed acoustic signals in directions other than through the imaging plane, such as to provide additional acoustic signals to detect the position of a sensor. To scatter a portion (e.g., 5%) of the beamformed signals into different directions, for example sideways under the skin surface, the probe 205 or certain components of the probe 205 can be modified or reconfigured. In some examples, the lens on the distal end of the probe can be designed and manufactured in certain shape or pattern for scattering a small portion of the beamformed acoustic signals sideways. In some examples, certain scattering particles (e.g., metal particles) or articles (e.g., thin lines of metal or glass) can be manufactured into the lens, so that the lens can scatter the beamformed ultrasound signals sideways. In some examples, a scattering layer (or medium) is added to the outer surface of the lens of the probe 205. When the probe 205 is touching the skin surface, the scattering layer is between the lens and the skin surface to scatter the ultrasound signals transmitted from the transducer of the probe 205. In some examples, a separate component configured to scatter acoustic signals can be clipped onto the distal end of the probe. In other embodiments, the probe emits an acoustic signal or beamformed transmission that may include a wider band width or multiple frequencies, that in conjunction with the configuration of the lens, enhance tracking. Additionally, this transmission may include a range of frequencies selected such that spatial broadening is frequency dependent during the transmission to enable separate optimization of the image reconstruction and the tracking.

Prior to the biopsy procedure, the ultrasound probe 205 or another probe scans a region of interest to generate multiple ultrasound images for consecutive slices of the region of interest. A slice of the region of interest corresponds to the imaging plane when the ultrasound probe is at a certain location. As the ultrasound probe moves, the imaging plane is moving, and another ultrasound image for the next slice of the region of interest can be obtained. A processor, for example processor 160 in the processing system 200 in FIG. 1, creates a 3D model of the region of interest and determines a location of the biopsy target 230 based on the ultrasound images.

Prior to inserting the needle through the skin surface 210, the ultrasound probe 205 transmits beamformed acoustic signals. As soon as the needle 225 touches the skin surface 210, it may begin receiving reflected acoustic signals in the tracking region 250. The processor 160 starts tracking the needle location and orientation (e.g., angle) based on data from the IMU and the sensor. The processor 160 determines an optimized insertion point based on the orientation of the needle and the location of the biopsy target 230. The processor 160 determines a projected trajectory path based on the insertion point and orientation of the needle 225 and the location of the biopsy target 230, using a linear regression algorithm. The processor can provide visualization of the projected trajectory for each potential insertion point via a display and also suggest the medical practitioner to adjust the insertion point or the orientation of the needle. For example, as illustrated in FIG. 2, if the needle (denoted as 225-1) inserts at point 240-1 with a specific angle (or slope), the tip of the needle would be outside the imaging plane 220 even though still in the directly insonified region, the acoustic SS received by the sensor 235-1 coupled to tip of the needle 225-1 would be weaker; the SNR is also weaker. Alternatively, if the needle inserts at point 240-2 with the same angle, the projected trajectory shows that the tip of the needle would be within the imaging plane 220, the SS received by the sensor 235-2 coupled to the tip of the needle 225-2 would be stronger, so is the SNR. Thus, the insertion point 240-2 would be a better option with the specific angle. If the needle is inserted at point 240-1, simple ToF, triangulation and coherent image techniques for tracking the needle may not work as well when the tip of the needle is outside the imaging plane 220 because of the weaker SNR/SS and competing reflections from the surrounding anatomy. However, the tracking methods based on the shortest distance detection, which will be described below, can still work effectively.

For example, a probe is in contact of the skin of a body region and transmits ultrasound signals to the body region, and an instrument (e.g., a needle) is inserted into the body region. A sensor (e.g., an optical sensor) coupled to the instrument receives ultrasound signals and converts the ultrasound signals to optical signals. The optical sensor transmits the optical signals to a processor (e.g., processor 160 in FIG. 1), for example via an optical fiber. The processor converts the optical signals to electrical signals, and uses the electrical signals to determine the location of the sensor, thereby the location of the instrument. For example, the processor determines ToF from the transducer elements in the probe to the optical sensor coupled to the needle to determine the optical sensor's location relative to the transducer.

In some examples, transducer elements in the ultrasound probe transmit beamformed acoustic signals through the tissue of the body region, and the optical sensor coupled to the instrument receives certain ultrasound signals, for example directly transmitted from the transducer or reflected from the body tissue. The optical sensor generates optical signals based on the received acoustic signals and transmits them to the processor. The optical signals may then be converted to electrical signals.

In some examples, the processor generates an image (which can be called ToF image), for example by plotting the measured energy received by the sensor at each time interval (row) for a given acoustic beamforming ray (column). Then, the processor measures the acoustic energy at the expected ToF for each possible position of the sensor. Assuming the optical sensor is at the position with the highest energy, the processor detects the location of the sensor (e.g., the tip of the needle where the optical sensor is coupled to) with the highest energy and provides a visualization of the tip of the needle on the image.

In some examples, the processor uses a trained artificial intelligence (AI) model to process the ToF image and/or the acoustic energy calculated with expected ToF for each possible position to detect the needle position. The input data is the image, and the output is the x, y, z coordinates relative to the transducer's center element. For example, the trained AI model includes convolutional neural networks (CNNs). The trained AI model is especially suitable to handle complex scenarios. For example, some ultrasound signals take a quicker path, for example propagation in needle (stainless steel has a higher speed of sound than the tissue does), than the straight line in tissue. This causes the signal path to deviate. However, the trained AI model can process the image and determine the location of the optical sensor accurately. Other suitable AI models may be used.

The processor alternatively may also use a trained AI model to integrate intra-procedural imaging with trajectory prediction algorithms that account for tissue resistance and anatomical variability. The system enables physicians to visualize and track the position and projected path of tools within soft tissues, even after active deployment (e.g., firing of a spring-loaded biopsy needle), where manual control is no longer feasible. By incorporating prior knowledge of tool-tissue interaction—including empirical data or models of how tools decelerate or deviate upon entering biological tissue, adaptable to the patient's current anatomical responses—example systems and methods according to this disclosure can optimize the initial position, orientation, and firing parameters to ensure accurate targeting of anatomical regions of interest.

In some examples, the processor uses the ultrasound's ToF to the sensor to fit the boundary of the ultrasound's arrival to a curve, which can be affected by probe geometry, speed of sound of tissue, and transmit delays. The processor then uses that curve's parameters to determine the sensor's position. First, the processor identifies the boundary of the ultrasound's arrival. The boundary can be identified using an edge enhancing filter (e.g., a Sobel filter), an edge detection method (e.g., canny edge detection), or by using Laplacian operators, Difference of Gaussians (DoG) operators, thresholding, etc. Second, the processor fits a curve to the detected boundary, for example by defining the boundary's expected shape and finding the best fit parameters using optimization techniques. Alternatively, the processor 160 fits a curve to the detected boundary by performing a multiscale template matching of an expected boundary.

In some examples, the processor preprocesses the ToF image. The preprocessing includes down-sampling and filtering to reduce noise in the ToF image. The preprocessing also includes applying intensity compression to enhance contrast of the ToF image. Contrast enhancement ensures meaningful structures in the ultrasound image remain visible. The preprocessing also includes suppressing bright reflections to minimize interference. The preprocessing further includes normalizing darker regions to improve overall visibility and reduce false positive edge detection.

The processor may then perform a history-based tracking with a search (e.g., a coarse-to-fine search) in the preprocessed ToF image. If a previous image frame provides a reliable needle location, the processor performs a quick or fine search by defining a small search window or range based on the prior location. Randomized small movements in the small search window increases robustness. In some examples, the processor 160 applies a weighting function, computes a fitting merit function, evaluates signal strength, and updates the location if the confidence is high.

If the quick search fails to detect the location of the optical sensor coupled to the needle, the processor performs a complete or coarse search in the entire ToF image. In some examples, the processor 160 uses a grid-based search to locate the optical sensor using ToF calculations. Multiple interactions are used to narrow down the search range and improve accuracy.

In some examples, the optical sensor receives reflected or scattered acoustic signals, besides the acoustic beamforming signals, which can also be used for tracking the location of the optical sensor. When reflected or scattered signals are used for tracking the optical sensor, the processor obtains an ultrasound image (e.g., B-mode image) of a region around the sensor. The B-mode image includes bright spots which can be the moving sensor that is coupled to the needle or certain tissue locations that reflect or scatter ultrasound signals. Such a bright tissue location can be considered as an “internal ultrasound transmitter.” The processor determines the (x, y, z) information of the “internal ultrasound transmitter” based on the B-mode image. The optical sensor receives acoustic signals from one or more “mini ultrasound transmitters,” converts the acoustic signals to optical signals, and transmits to the processor. The processor can determine ToF information based on data from the optical sensor and the location of the “mini ultrasound transmitters,” and determines the location of the optical sensor using the shortest distance method, generally as described above.

Once the processor detects a location of the optical sensor, the processor computes the SNR/SS of the location to evaluate the fitting wellness (e.g., the confidence level for the fitting) and the signal strength at the detected location. A higher SNR/SS indicates a more reliable and confident fit.

In some examples, the processor determines ToF information (e.g., arrival time) for the ultrasound signals received at the optical sensor based on the optical signals received by the processor from the optical sensor. A probe can sweep the skin of a body region to transmit acoustic beamforming signals from different angles relative to the skin, the user can vary the angle of the probe to achieve a similar effect. The optical sensor coupled to an instrument in the body region receives acoustic signals and generates corresponding optical signals, which are then sent to a processor. The processor can then convert the optical signals to electrical signals as sensor data. The processor can use signal processing techniques to detect the ToF (or arrival time) of the ultrasound signals received at the optical sensor.

In addition, multiple sets of ToF data can be pre-determined for multiple locations of the body region. In some examples, an ultrasound image is generated for the body region based on the acoustic signals received by the transducer elements in the probe. A set of ToF data corresponding to each pixel of the ultrasound image can be pre-determined. The ToF data is precomputed based on transmit delays, geometrical calculation of the transmit wavefront, and speed of sound of tissue. Each pixel in the ultrasound image corresponds to a location (x, y) in the body region shown in the ultrasound image. The set of ToF data at each pixel location can be visualized as a corresponding ToF curve. The processor compares the ToF information associated with the optical sensor to the pre-determined ToF data corresponding to different locations of the body region (e.g., different pixels of the ultrasound image of the body region) to identify the set of pre-determined ToF data that matches the ToF information associated with the optical sensor. Then, the location of the optical sensor can be determined to correspond to the location with the matching pre-determined ToF data.

For example, the processor generates a curve based on the ToF data associated with the signals from the optical sensor. Meanwhile, multiple curves are also generated based on the pre-determined ToF data associated with multiple locations in the body region. The processor performs a curve search or curve fitting to identify a curve corresponding to a set of pre-determined ToF data that fits the curve corresponding to the ToF data associated with the optical sensor.

To perform the curve search, the processor may use a brute-force search by checking every pre-computed curve against the curve based on the sensed ToF data, in some examples. In other examples, it may use a more targeted search, such as based on a prior determined location of the sensor and a pre-determined threshold search region surrounding the prior determined location. The size of the search region may vary based on information received from the IMU, such as the speed at which the instrument is being inserted into the tissue. In some examples, it may initially use a brute-force search and then transition to a more targeted search once the location of the tip of the instrument has been determined or after multiple successive locations have been determined.

A curve corresponding to ToF data associated with a location in the body region or a pixel in an ultrasound image of the body region may have a greater or lesser magnitude depending on whether the location is in-plane or not with respect to the beamformed acoustic signals from the probe. When the sensor is out of plane, it will receive less acoustic energy, reducing the magnitude of the corresponding curve representing the ToF data; however, the curve shape remains essentially the same. Therefore, the processor not only can determine both the correct location of the sensor on the ultrasound image, but also whether the sensor is in-plane or out of plane based on the magnitude of the curve. The processor can provide a visual indicator when it is out of plane. In some examples, the processor can determine how far it is out of plane based on the SS received at the optical sensor.

In some examples, the processor pre-processes the signal ToF data. The preprocessing includes down-sampling, filtering, and other denoising measures to reduce noise in the signal ToF data. The preprocessing also includes signal conditioning to further enhance the ToF curve. The preprocessing further includes an edge detection step to detect the shape of the curve, and in some examples, the edge detected signal image is normalized and weighted based on pre-determined weight of the transmitted acoustic energy.

The processor may then perform a curve matching based on pre-computed curve shapes informed by transducer geometry, acoustic media, and transmit delays. Multiple candidate curves are compared against the curve representing the acoustic data associated with the sensor to select the most likely location of the instrument.

Once the processor obtains a location of the instrument, the processor uses the signal strength of the location to evaluate the fitting quality (e.g., the confidence level for the fitting). A higher signal strength and better match with the precomputed curves indicates a more confident observation, thus a higher instrument location confidence.

In some examples, the processor visualizes the detected location in a display. The display also shows the projected trajectory of the needle based on the location of the optical sensor coupled to the needle and the target location, allowing the practitioner to determine when the correct trajectory has been reached. In some examples, the processor also provides guidance to the medical practitioner about how to adjust the location or orientation of the needle, for example via text, graphics, or audio, The processor stores data related to the detected location in the current image frame and uses it to search for the location of the optical sensor coupled to the needle in subsequent image frames.

The location tracking techniques in the present disclosure are robust in challenging environments and can still provide reliable tracking near bones, air pockets, and reflective surfaces and when the SNR/SS varies within the tracking region. The present location tracking techniques applicable in extended needle detection range. For example, a processor can detect the needle location as soon as it touches skin surface before insertion. Thus, these techniques can assist in pre-procedure trajectory planning. Meanwhile, the present techniques can minimize jitter and improve real-time tracking consistency with higher accuracy and stability. With the adaptive search algorithm and parallel processing, the present techniques provide faster computation and processing speed for location detection. The present techniques can significantly improvs needle localization, by leveraging ToF for more accurate tracking. The integration with the needle visualization system can enhance real-time needle guidance, making procedures more precise, reliable, and efficient.

In some examples, the processor generates an ultrasound image depicting a field of view for the sensor coupled to the instrument, which can be called an OnPoint image. An ultrasound transducer generates a focused beam to insonicate a portion of body tissue or a body region of interest. The boundaries and scatterers along the insonication beam path can reflect, refract, or scatter ultrasound signals. The time delay to the reflective boundaries or scatters are determined by the relative locations of the boundaries or scatterers to the transducer and the transmit wave. The reflected, refracted, and scattered ultrasound signals travel in a direct path to the sensor and are received by the sensor. The time delay is determined by the relative location of the boundaries or scatterers to the sensor.

For a location of interest in the body region, the time delay (e.g., T1) from the transmission to the location of interest and the time delay (e.g., T2) from the location of interest to the sensor are calculated. The portion of sensor-detected signal corresponding to the total time delay (e.g., T1+T2) is used to calculate the scatterer intensity of the location of interest. This process is repeated for one or more location of interest inside the insonicated region. This process can also be repeated for one or more insonicated regions corresponding to one or more transmit events to reconstruct the OnPoint image. Thus, the system is able to determine the relative position of the locations of interest with respect to the sensor based on the timing data. Thus, their position within an image from the perspective of the sensor can be determined. The OnPoint reconstruction process can also include filtering, interpolation, envelope detection, and compression to form an image. The image is then scan-converted (or rasterized) to polar coordinates to correspond to the scan geometry of the sensor.

Enhanced location accuracy and tracking permitted by on-tool optical-based ultrasound transducers can facilitate and improve this technique by increasing the ability to visualize and track the ablation tool and visualize the anatomy from the perspective of the sensor (e.g., OnPoint imaging), as well as providing real time ablation monitoring for the ablation procedure.

For example, the thyroid is a relatively small portion of tissue with many adjacent structures that an interventionalist tries not to damage; e.g., the vagus nerve, the esophagus, the common carotid artery, the inferior jugular vein, the anterior jugular vein, the trachea, and the so-called “danger triangle” including the recurrent laryngeal nerve. Careful and accurate location of the ablation tool, and imaging of the surrounding anatomy from the perspective of the sensor (OnPoint imaging), facilitated by an on-tool optical-based ultrasound sensor, can aid the interventionalist in avoiding these areas by monitoring the ablation progression in real time.

OnPoint imaging (i.e., creating ultrasound images from the perspective of the sensor location in situ, such as, but not limited to, B-mode, color doppler, shear wave, PW doppler, etc.) relies on the sensor being able to receive the beamformed acoustic signals, The beamformed signals may be generated from a variety of probes, including externally positioned ultrasound probes to transesophageal echocardiography (TEE) probe as the transmit source for OnPoint imaging for image quality better or from a different anatomical locations than an ultrasound excitation from outside the body. Signal deconvolution approaches may be used to reduce internal ultrasound propagation, improving the signal homogeneity for OnPoint imaging.

For this type of multi-modality imaging, the all-fiber-based OnPoint imaging could offer distinct advantages over ultrasound excited externally or from IVUS, ICE, or TEE transducers. Acoustic lenses and/or mechanical spiral/steering structures may be used to scan the sensor through a region of interest to form an image, and multi-modality co-registration can combine functional information of the tissue, enhancing imaging and/or characterization. Multi-modality imaging with IVUS, ICE or TEE also provides advantages by incorporating OnPoint imaging from the position of the sensor or sensors to supplement and expand upon the imaging from such transducers. This may also provide imaging in areas where such transducers have limitations, such as by allowing beamforming through acoustic challenging regions (e.g., ribs) with the OnPoint acoustic geometry.

OnPoint imaging could be used to identify calcification on valves, particularly by detecting a strong reflection of a calcified valve, thus making calcifications easy to differentiate from valve/tendons. OnPoint can also be used to identify the valve location for, as a non-limiting example, rough placement of TAVR, TMVR or TEE to reduce fluoroscopy time. The use of this ultrasound-based system reduces radiation exposure for physicians.

In some examples, OnPoint doppler may be used for regurgitation detection, as a non-limiting example. Regurgitation after valve placement is a routine check for TAVR, TMVR or TEE and using OnPoint doppler provides the ability to pick up lower velocity regurgitations. The imaging frame rate could be increased for more at-valve visualization by skipping the tracking and/or B-mode frame. The frame rate requirement in heart ultrasound imaging is higher due to heart motion, and skipping the B-mode and tracking frame allows for a faster refresh rate. Additionally, acquiring only the signal from the sensor time of arrival to the region of interest being visualized can significantly increase the frame rate. As another example, shear wave imaging also requires a high frame rate. The shear wave created by the patient's beating heart could be used for imaging. The measurement of myocardial stiffness can provide insights into cardiac pathophysiology. As a further alternative, a directional fiber sensor could also be used for shear wave enhancement. It should be understood that the various sensors discussed above may be used in combination with, or integrated with, any suitable optical or acousto-optic components, such as acoustic lenses and the like.

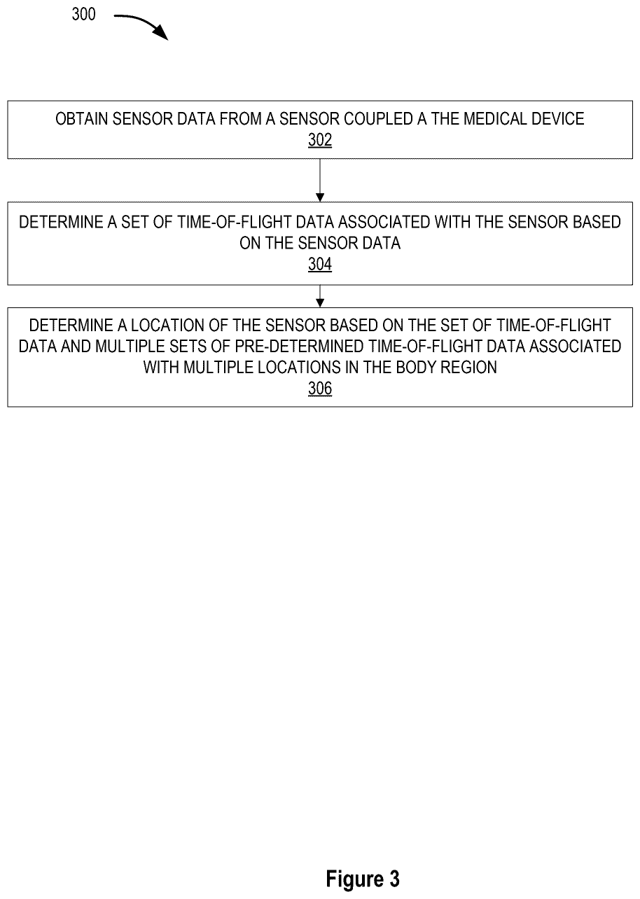

Turning to FIG. 3, FIG. 3 shows an example method of tracking the location of a medical instrument during a medical procedure, according to this disclosure. The example method 300 will be discussed with respect to system 101 shown in FIG. 1; however, any suitable system for ultrasound visualization and tracking of a medical device may be used.

At step 302, a processor 160 obtains sensor data from a sensor 125 coupled to a medical device. Transducers from probe 115 transmit a set of acoustic signals, such as beamformed acoustic signals, to a body region (e.g., medium 105). The sensor 125 receives acoustic signals, which may include reflections of the set of acoustic signals transmitted by the probe 115, in the body region and converts the received acoustic signals to the sensor data. For example, the sensor 125 is an optical sensor. It converts the received acoustic signals to optical signals. The processor 160 receives the optical signals and converts the optical signals to electrical signals for further processing.

At step 304, the processor 160 determines a set of ToF data associated with the sensor 125 based on the sensor data. The processor 160 processes the sensor data using signal processing techniques to determine the ToF or arrival time of the ultrasound signal received by the sensor 125. In some examples, the processor generates a curve based on the set of ToF data associated with the sensor 125. Similarly, the processor 160 pre-generates multiple curves based on the multiple sets of ToF data, corresponding to multiple locations in the body region, generally as discussed above with respect to FIG. 2.

At step 306, the processor 160 determines a location of the sensor 125 based on the set of ToF data and multiple sets of pre-determined ToF data associated with multiple locations in the body region. In some examples, the processor 160 compares the set of ToF data with the multiple sets of pre-determined ToF data to identify a set of pre-determined ToF data corresponding to a location in the body region that best matches the set of ToF data associated with the sensor. The best-matched set of pre-determined ToF data and the set of ToF data associated with the sensor have the least deviation from each other. The location corresponding to the set of pre-determined ToF data is the location where the sensor is in the body region.

The processor 160 performs a curve fitting process, to fit the curve representing the ToF data associated with the sensor to the pre-generated curves representing the multiple sets of pre-determined ToF data corresponding to multiple locations of the body region. The processor 160 identifies a pre-generated curve that best matches the curve representing the set of ToF data associated with the sensor. The location in the body region corresponding to the pre-generated curve that best matches the curve associated with the sensor is the determined location of the sensor in the body region. The location of the sensor can then be highlighted on the B-mode image, such as by displaying a colored spot corresponding to the determined location.

In some examples, the processor generates an ultrasound image (e.g., B-mode image) depicting the view of the probe. The processor can reconstruct the ultrasound image based on the location of the sensor in the body region to generate an ultrasound image (e.g., OnPoint image in B-mode) depicting the view of the sensor.

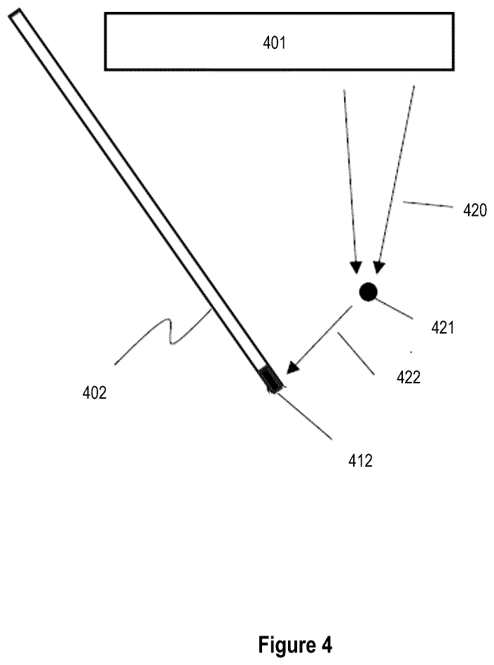

Turning to FIG. 4, FIG. 4 shows an optical sensor coupled to a needle receiving a reflected ultrasound signal from a tissue spot. In FIG. 4, a needle 402 is inserted into the body tissue. An optical sensor 412 is coupled to the tip of the needle 402. An ultrasound probe 401 transmits ultrasound beamforming signals 420 to the body tissue under the skin. A tissue spot 421 reflects some of the ultrasound beamforming signals 420, and becomes ultrasound signal 422. The tissue spot 421 can be considered as an “internal ultrasound transmitter.” A processor can determine a location of the “internal ultrasound transmitter” based on an ultrasound image of the region (e.g., B-mode image from the perspective of the ultrasound probe 401). The optical sensor 412 receives the ultrasound signal 422. As described above, a processor can determine a location of the optical sensor 412, using the shortest distance method, based on ToF information associated with reflected ultrasound signals (e.g., ultrasound signal 422) and the location of the “internal ultrasound transmitters.”

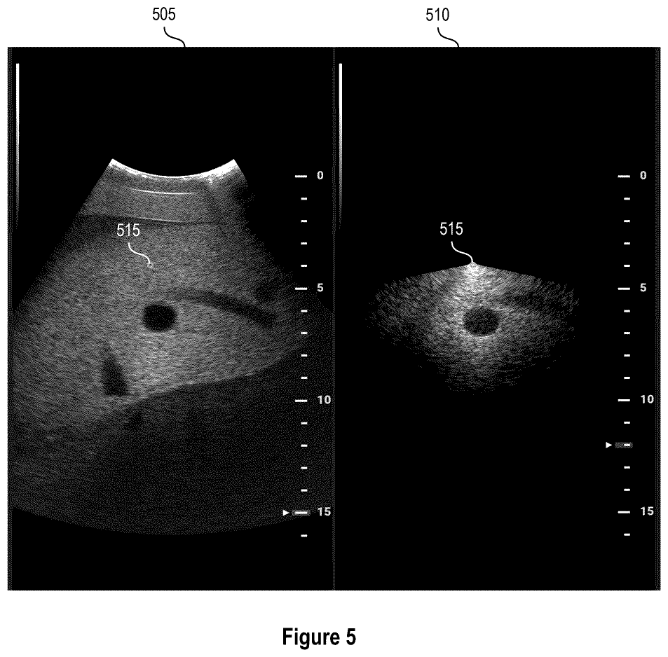

Turning to FIG. 5, FIG. 5 shows an ultrasound image depicting the view of a body region from the perspective of a probe 115 and an OnPoint image 510 depicting the view of the body region from the perspective of a sensor 515 in the body region, according to this disclosure. A processor can determine the location of the sensor 515 using the techniques described above. The processor then reconstructs the ultrasound image 505 to generate the OnPoint image 510 based on the location of the sensor 515 using the reconstruction techniques described above. The ultrasound image 505 and the OnPoint image 510 register the sensor 515 at the same depth.

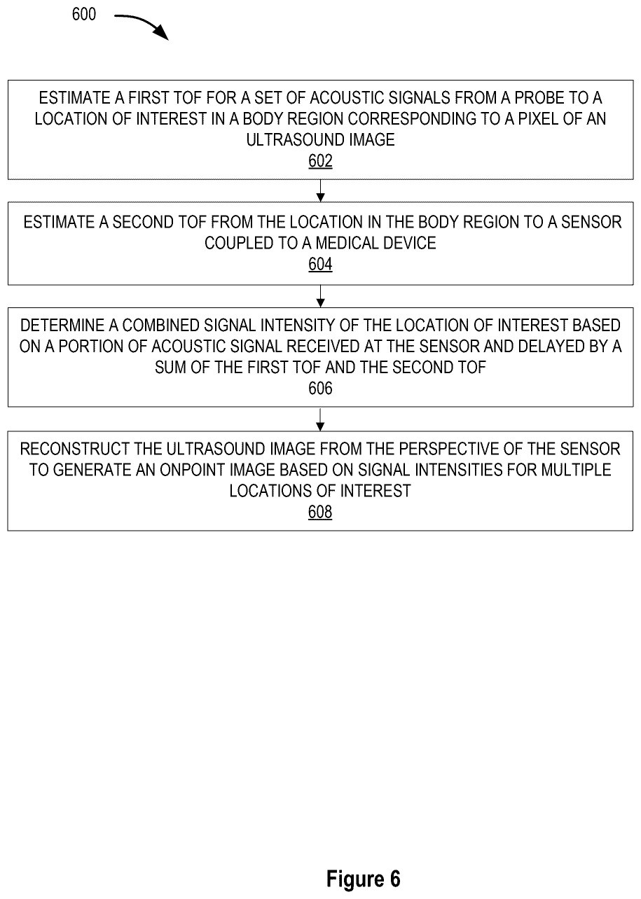

Turning to FIG. 6, FIG. 6 shows an example method of reconstructing an ultrasound image to generate an OnPoint image according to the present disclosure. The example method 600 will be discussed with respect to system 101 shown in FIG. 1; however, any suitable system for ultrasound visualization and tracking of a medical device may be used.

At block 602, a processor estimates a first ToF for a set of acoustic signals from a probe to a location of interest in a body region corresponding to a pixel of an ultrasound image. The probe transmits the set of acoustic signals to the body region. The ultrasound image is pre-generated for the body region from the probe's perspective. The first ToF is estimated based on the distance between the probe and the location of interest and the speed of acoustic signals in the body region.

At block 604, the processor estimates a second ToF from the location in the body region to a sensor coupled to a medical device. The location of the sensor is pre-determined, for example based on FIG. 3. The second ToF is estimated based on the distance between the location of interest and the location of the sensor and the speech of acoustic signals in the body region.

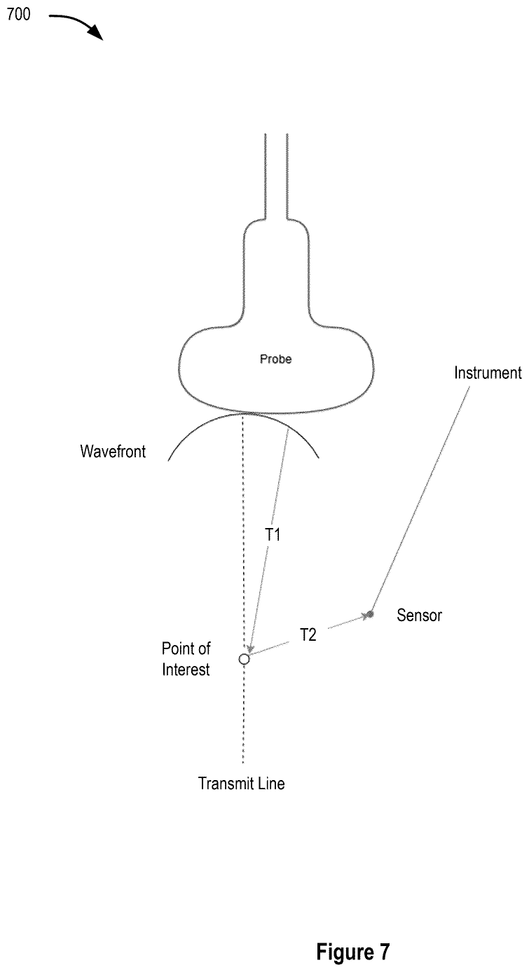

At block 606, the processor determines a combined signal intensity of the location of interest based on a portion of the acoustic signal received by the sensor and delayed by a sum of the first ToF and the second ToF, for example as shown in FIG. 7. This delay and sum approach is similar to conventional ultrasound imaging approach but is implemented for singular points instead of for receiving PZT AE arrays.

At block 608, the processor reconstructs the ultrasound image from the perspective of the sensor to generate an OnPoint image based on signal intensities for multiple locations of interest, which can be determined based on block 606.

Turning to FIG. 7, FIG. 7 illustrates an example of ToF delay calculation for a location of interest. The location of the wavefront of a beamformed signal from the probe is used to calculate the ToF T1 of the beamformed signal to the location of interest. The transmit line in FIG. 7 shows the direction of the beamformed signal. The beamformed signal is reflected at the location of interest. The location of interest's relative location to a sensor coupled to an instrument is used to calculate the ToF T2 of the reflected acoustic signal.

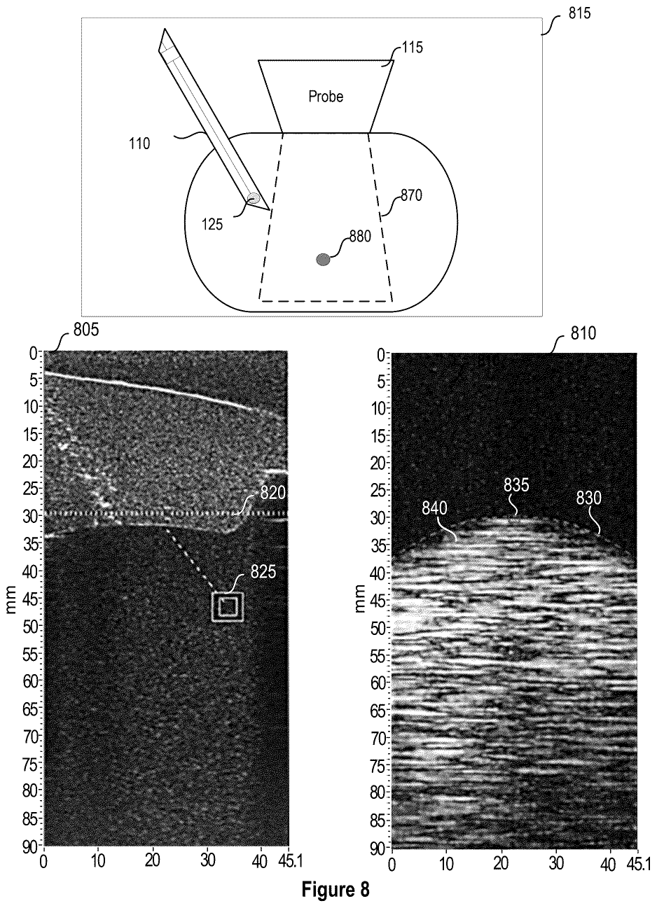

Turning to FIG. 8, FIG. 8 shows a B-mode image 805 and a ToF image 810 in a first instance during an example medical procedure. The first instance of the example medical procedure is illustrated in diagram 815, with the two images 805, 810 generated in real-time, corresponding to the position and orientation of the needle 110 shown in diagram 815.

An ultrasound probe 115 transmits beamformed acoustic signals and also receives echo acoustic signals. An ultrasound image (or images), for example the B-mode image 805, may be generated based on echo acoustic signals. The B-mode image 805 is from the perspective of the ultrasound probe 115. The probe head is the point of origin, and the image 805 depicts objects (e.g., tissue) arranged in the field of view of the probe. The horizontal line 820 represents the locations of the needle 110, relative to the target location 880. The double box 825 in image 805 represents projected destination of the needle 110 based on the current location and orientation. The double box 825 includes an outer box and an inner box, with the outer box indicating where the needle 110 will cross the imaging plane 870. As the needle 110 is moving, rotating, or tilting, the size of the inner box may change. If the inner box grows bigger, it indicates that the trajectory of the needle 110 is moving closer to the target location 880. An optical sensor 125 coupled to the needle 110 also receives acoustic signals. An ultrasound image, for example ToF image 810, can be generated from the acoustic signals received by the optical sensor 125 coupled to the needle 110. The ToF image 810 is from the perspective of the optical sensor 125 and the image represents objects arranged in the field of view of the optical sensor 125. The point of origin is within the body of a patient. The curve 830 in image 810 is the fitted curve for the boundary of the ultrasound's arrivals. The small box 835 on curve 830 is the determined location of the optical sensor 125 coupled to the needle 110. In some examples, two different fitted curves, for example curve 830 and 840, are generated, for example by running the shortest distance algorithm in parallel. The location of the optical sensor 125 can be determined between the two fitted curves.

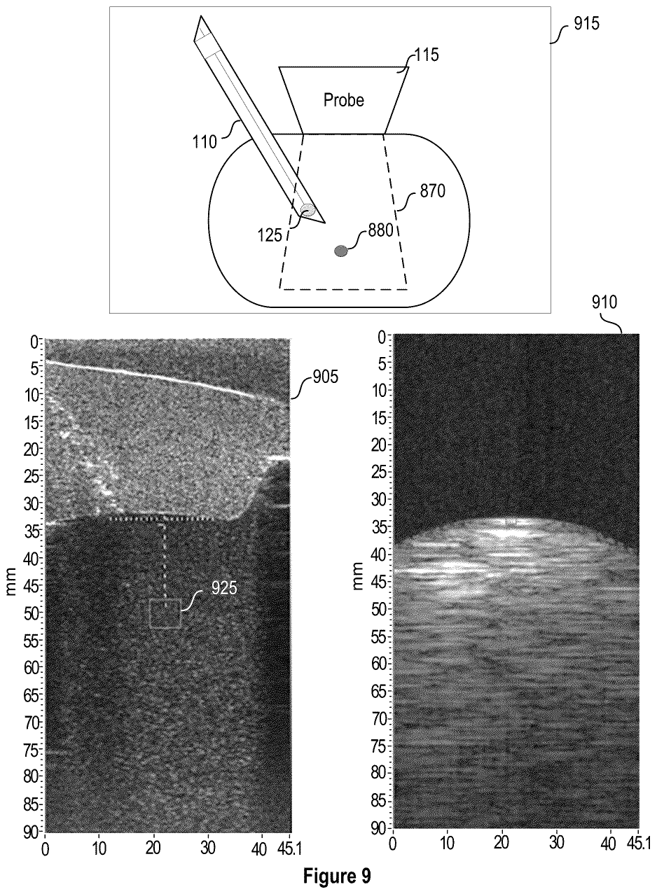

Turning to FIG. 9, FIG. 9 shows a B-mode image 905 and a ToF image 910 in a second instance during an example medical procedure. Similar to the images in FIG. 8 corresponding to the first instance of the example medical procedure, the second instance of the example medical procedure is illustrated in diagram 915, with the two images 905, 910 generated in real-time, corresponding to the position and orientation of the needle 110 shown in diagram 915. As described in FIG. 9, the inner box of the double box 925 gets bigger as the trajectory of the needle 110 is moving closer to the target location 880. When the trajectory of the needle 110 overlaps with the target location 880 (the target location 880 is on the trajectory of the needle and in the imaging plane) in the second instance, the inner box is as big as the outer box, and it turns green, illustrated as box 925 in FIG. 9.

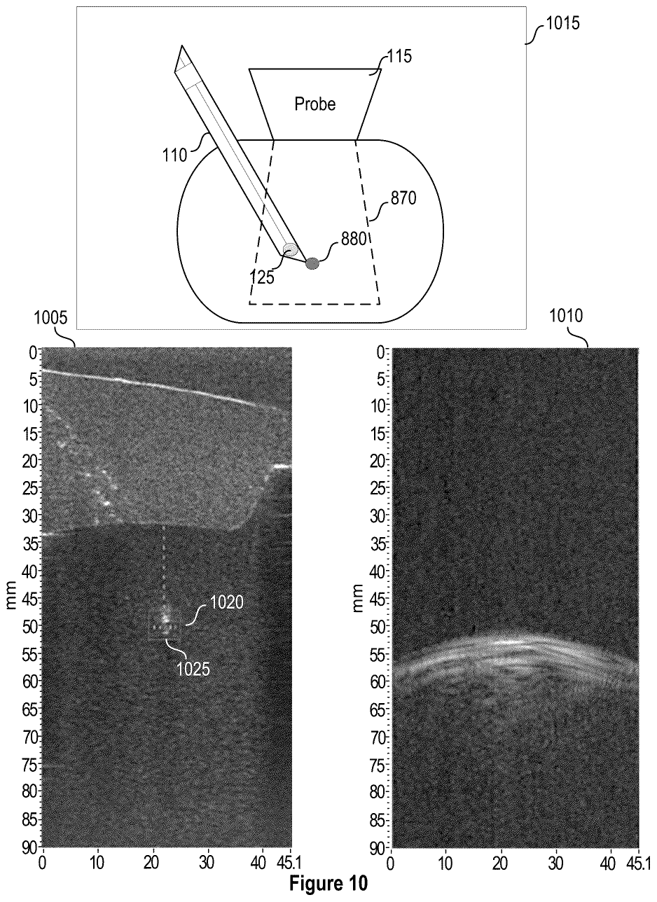

Turning to FIG. 10, FIG. 10 shows a B-mode image 1005 and an ToF image 1010 in a third instance during an example medical procedure. Similar to the images in FIG. 8 corresponding to the first instance of the example medical procedure, the third instance of the example medical procedure is illustrated in diagram 1015, with the two images 1005, 1010 generated in real-time, corresponding to the position and orientation of the needle 110 shown in diagram 1015. As described in FIG. 9, in the second instance, the trajectory of the needle 110 overlaps with the target location 880 (the target location 880 is on the trajectory of the needle 110), the needle 110 continues to move toward the target location 880. In the third instance, the needle 110 reaches the target location 880, the horizontal line 1020 which indicates the location of the needle 110 overlaps with the green box 1025, which represents the target location 880.

The foregoing description of example embodiments has been presented only for the purpose of illustration and description and is not intended to be exhaustive or to limit the disclosure to the precise forms disclosed. Numerous modifications and adaptations thereof will be apparent to those skilled in the art without departing from the spirit and scope of the disclosure.

Reference herein to an embodiment, example, or implementation means that a particular feature, structure, operation, or other characteristic described in connection with the example may be included in at least one implementation of the disclosure. The disclosure is not restricted to the particular examples or implementations described as such. The appearance of the phrases “in an embodiment,” “in one example,” “in an example,” “in one implementation,” or “in an implementation,” or variations of the same in various places in the specification does not necessarily refer to the same example or implementation. Any particular feature, structure, operation, or other characteristic described in this specification in relation to one example or implementation may be combined with other features, structures, operations, or other characteristics described in respect of any other example or implementation.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and A and B and C.

What is claimed is:

1. A method of tracking a medical device in a medical procedure, comprising:
obtaining sensor data from a sensor coupled to the medical device in a body region, wherein the sensor receives one or more acoustic signals and converts the one or more acoustic signals to the sensor data, the one or more acoustic signals being emitted by a transducer;
determining a set of time-of-flight (ToF) data associated with the sensor based on the sensor data; and
determining a location of the sensor based on the ToF data set and multiple sets of pre-determined ToF data associated with multiple locations in the body region.

2. The method of claim 1, wherein the sensor comprises an optical sensor, and
wherein the optical sensor converts the one or more acoustic signals to optical signals, and wherein a processor further converts the optical signals to electrical signals.

3. The method of claim 1, further comprising:
comparing the set of ToF data associated with the sensor with the multiple sets of pre-determined ToF data to identify a set of pre-determined ToF data that matches the set of ToF data associated with the sensor; and
determining that the location of the sensor corresponds to a location associated with the set of pre-determined ToF data.

4. The method of claim 1, further comprising:
generating multiple curves corresponding to the multiple sets of pre-determined ToF data associated with the multiple locations in the body region to obtain multiple pre-computed curves;
generating a curve corresponding to the set of ToF data associated with the sensor;
fitting the curve to the multiple pre-computed curves to identify a pre-computed curve that matches the curve; and
determining that the location of the sensor corresponds to a location associated with the pre-computed curve.

5. The method of claim 1, further comprising:
causing the transducer to transmit a set of beamformed acoustic signals;
generating an ultrasound image based on acoustic echo signals received by the transducer, wherein the acoustic echo signals are based on the set of beamformed acoustic signals, and wherein the ultrasound image depicts a view from the transducer; and
overlaying the location of the sensor on the ultrasound image.

6. The method of claim 5, further comprising:
generating a second ultrasound image based on the ultrasound image and the location of the sensor, wherein the second ultrasound image depicts a view from the sensor.

7. The method of claim 1, further comprising:
determining a relative location of the sensor in reference to an imaging plane based on signal strength of the sensor data, wherein the imaging plane is associated with beamformed acoustic signals transmitted from the transducer.

8. A system comprising:
a communications interface;
a non-transitory computer-readable medium; and
one or more processors communicatively coupled to the communications interface and the non-transitory computer-readable medium, the one or more processors configured to execute processor-executable instructions stored in the non-transitory computer-readable medium to:
obtain sensor data from a sensor coupled to a medical device in a body region, wherein the sensor receives one or more acoustic signals and converts the one or more acoustic signals to the sensor data, the one or more acoustic signals being emitted by a transducer;
determine a set of time-of-flight (ToF) data associated with the sensor based on the sensor data; and
determining a location of the sensor based on the ToF data set and multiple sets of pre-determined ToF data associated with multiple locations in the body region.

9. The system of claim 8, wherein the sensor comprises an optical sensor, and
wherein the optical sensor converts the one or more acoustic signals to optical signals, and wherein a processor further converts the optical signals to electrical signals.

10. The system of claim 8, wherein the one or more processors are configured to
execute further processor-executable instructions stored in the non-transitory computer-readable medium to:
compare the set of ToF data associated with the sensor with the multiple sets of pre-determined ToF data to identify a set of pre-determined ToF data that matches the set of ToF data associated with the sensor; and
determine that the location of the sensor corresponds to a location associated with the set of pre-determined ToF data.

11. The system of claim 8, wherein the one or more processors are configured to
execute further processor-executable instructions stored in the non-transitory computer-readable medium to:
generate multiple curves corresponding to the multiple sets of pre-determined ToF data associated with the multiple locations in the body region to obtain multiple pre-computed curves;
generate a curve corresponding to the set of ToF data associated with the sensor;
fit the curve to the multiple pre-computed curves to identify a pre-computed curve that matches the curve; and
determine that the location of the sensor corresponds to a location associated with the pre-computed curve.

12. The system of claim 8, wherein the one or more processors are configured to
execute further processor-executable instructions stored in the non-transitory computer-readable medium to:
cause the transducer to transmit a set of beamformed acoustic signals;
generate an ultrasound image based on acoustic echo signals received by the transducer, wherein the acoustic echo signals are based on the set of beamformed acoustic signals, and wherein the ultrasound image depicts a view from the transducer; and
overlay the location of the sensor on the ultrasound image.

13. The system of claim 12, wherein the one or more processors are configured to
execute further processor-executable instructions stored in the non-transitory computer-readable medium to:
generate a second ultrasound image based on the ultrasound image and the location of the sensor, wherein the second ultrasound image depicts a view from the sensor.

14. The system of claim 8, wherein the one or more processors are configured to
execute further processor-executable instructions stored in the non-transitory computer-readable medium to:
determine a relative location of the sensor in reference to an imaging plane based on signal strength of the sensor data, wherein the imaging plane is associated with beamformed acoustic signals transmitted from the transducer.

15. A non-transitory computer-readable medium comprising processor-executable instructions configured to cause one or more processors to:
obtain sensor data from a sensor coupled to a medical device in a body region, wherein the sensor receives one or more acoustic signals and converts the one or more acoustic signals to the sensor data, the one or more acoustic signals being emitted by a transducer;
determine a set of time-of-flight (ToF) data associated with the sensor based on the sensor data; and
determine a location of the sensor based on the ToF data set and multiple sets of pre-determined ToF data associated with multiple locations in the body region.

16. The non-transitory computer-readable medium of claim 15, further
comprising processor-executable instructions configured to cause one or more processors to:
compare the set of ToF data associated with the sensor with the multiple sets of pre-determined ToF data to identify a set of pre-determined ToF data that matches the set of ToF data associated with the sensor; and
determine that the location of the sensor corresponds to a location associated with the set of pre-determined ToF data.

17. The non-transitory computer-readable medium of claim 15, further
comprising processor-executable instructions configured to cause one or more processors to:
generate multiple curves corresponding to the multiple sets of pre-determined ToF data associated with the multiple locations in the body region to obtain multiple pre-computed curves;
generate a curve corresponding to the set of ToF data associated with the sensor;
fit the curve to the multiple pre-computed curves to identify a pre-computed curve that matches the curve; and
determine that the location of the sensor corresponds to a location associated with the pre-computed curve.

18. The non-transitory computer-readable medium of claim 15, further
comprising processor-executable instructions configured to cause one or more processors to:
cause the transducer to transmit a set of beamformed acoustic signals;
generate an ultrasound image based on acoustic echo signals received by the transducer, wherein the acoustic echo signals are based on the set of beamformed acoustic signals, and wherein the ultrasound image depicts a view from the transducer; and
overlay the location of the sensor on the ultrasound image.

19. The non-transitory computer-readable medium of claim 18, further
comprising processor-executable instructions configured to cause one or more processors to:
generate a second ultrasound image based on the ultrasound image and the location of the sensor, wherein the second ultrasound image depicts a view from the sensor.

20. The non-transitory computer-readable medium of claim 15, further
comprising processor-executable instructions configured to cause one or more processors to:
determine a relative location of the sensor in reference to an imaging plane based on signal strength of the sensor data, wherein the imaging plane is associated with beamformed acoustic signals transmitted from the transducer.
